(19) United States
(12) Patent Application Publication
(10) Pub. No.: US 2023/0138189 A1
(43) Pub. Date: May 4, 2023

(54) INFUSION SYSTEM

(71) Applicant: B. Braun Melsungen AG, Melsungen (DE)

(72) Inventors: Hans-Christian Moritzen, Kassel (DE); Dennis Obermann, Willingshausen (DE); Thomas Wiegand, Homberg (DE)

(21) Appl. No.: 17/974,735

(22) Filed: Oct. 27, 2022

(30) Foreign Application Priority Data
Oct. 28, 2021 (DE) 10 2021 212211.6

(51) Int. Cl. A61M 5/168; A61M 5/142

(52) U.S. Cl. CPC A61M 5/16854; A61M 5/14228; A61M 2205/3331; A61M 2205/18; A61M 2005/16863

(57) ABSTRACT

An infusion system includes an infusion pump configured to convey a fluid to be administered through a fluid conduit, a number of sensors for producing associated sensor data, and an AI unit configured to calculate a number of target variables depending on the number of sensor data using a statistical model as a basis, the statistical model having been trained using training data.

### CROSS-REFERENCE TO RELATED APPLICATION

This application claims priority under 35 U.S.C. § 119 to German Application No. 102021212211.6, filed Oct. 28, 2021, the content of which is incorporated by reference herein in its entirety.

### FIELD

The present disclosure relates generally to infusion systems, and in particular to an infusion system that has increased operational safety.

### BACKGROUND

The goal of infusion therapy lies in a continuous administration of fluids over a certain amount of time. As a rule, this happens intravenously, arterially, subcutaneously or intraosseously via a catheter. Additionally, there also is parenteral feeding by means of infusion pumps.

Infusion pumps are used for great metering accuracy in device-based infusion technology. Known manifestations of infusion pumps include syringe pumps and peristaltic pumps, for example. Various operational faults may arise during the application using these pumps. One example is the occlusion of the fluid conduit and/or patient access. This operational fault is also referred to as occlusion and leads to the conveying process being interrupted. Since this may lead to a significant risk to the patient, the detection of an occlusion is within the fixed functional scope of conventional infusion equipment.

Conventional infusion systems usually used fixed threshold values for detecting an occlusion.

However, in the case of the lowest infusion rates, it may take several hours before an occlusion is identified. Timely identification of an occlusion may however be particularly relevant at these low rates since very effective medicaments are frequently administered at these rates.

The combination of low rates and, at the same time, very potent medicaments should be considered critical in the case of an occlusion in the previous, commercially available infusion equipment.

A problem lies in the fact that the use of very low threshold values is particularly difficult in the case of the conventional methods as said very low threshold values are accompanied by a high false alarm rate. Reasons can be found in various disturbance variables which are superposed on the relevant sensor signal (pressure/force), examples including the transition from static friction to dynamic fiction in the case of a syringe pump (slip-stick effect) and general variations in the nature of the plastic disposables.

### SUMMARY

The infusion system according to the present disclosure comprises a conventional infusion pump, which is configured to convey a fluid to be administered through a fluid conduit. By way of example, the infusion pump can be a syringe pump or a peristaltic pump.

The infusion system further comprises a number of sensors for producing associated sensor data. The number of sensors may be between 1 and 20, for example.

The infusion system further comprises an AI unit configured to calculate a number of target variables depending on the number of sensor data using a statistical model as a basis, the statistical model having been trained using training data. The number of target variables may be between 1 and 10, for example.

In an embodiment, the number of sensors are selected from the set of sensors consisting of: pressure sensors configured to produce sensor data in the form of pressure data in the fluid conduit of the fluid to be administered, force sensors configured to produce sensor data in the form of force data of a pump force produced by means of the infusion pump, motor current sensors configured to produce sensor data in the form of motor current data of an electric drive motor of the infusion pump and position sensors configured to produce sensor data in the form of position data of a syringe piston of the infusion pump.

In an embodiment, the number of target variables are selected from the set of target variables consisting of: an absolute pressure in the fluid conduit, a probability of an occlusion in the fluid conduit and a location of the occlusion in the fluid conduit.

In an embodiment, the AI unit is configured to calculate the number of target variables further depending on medicament data, patient data, data of further infusion pumps, therapy data, ambient data and/or vital parameter data using the statistical model as a basis.

In an embodiment, the infusion system further comprises an alarm unit configured to verify on the basis of the target variables whether an alarm condition has been satisfied and generate an alarm should the alarm condition have been satisfied.

In an embodiment, the infusion system further comprises a correlation analysis unit configured to calculate weight factors on the basis of all or some of the sensor data and/or on the basis of all or some of the target variables, the alarm unit being configured to verify on the basis of the weight factors whether the alarm condition has been satisfied.

In an embodiment, the alarm unit is configured to stop the infusion pump when the alarm condition occurs.

According to the present disclosure, the relevant sensor data, for example relating to a pressure produced by means of the pump and/or a pump force produced by means of the pump, and optionally further data or input variables, which allow the inference of an occlusion, are evaluated by means of the AI unit. According to the present disclosure, these input variables are evaluated by way of pattern recognition for the purposes of identifying an occlusion. The AI unit may be an integrated constituent part of the infusion pump. The AI unit may carry out a non-self-learning AI algorithm.

The AI unit preferably determines three target variables for identifying an occlusion, specifically an absolute or real pressure in the fluid conduit, a probability of an occlusion and a location of the occlusion.

The present disclosure initially renders it possible to be able to determine a first target variable in the form of a real fluid pressure in the fluid conduit from the sensor data to be processed, for example in relation to a measured pressure or a measured force, with an alarm being able to be generated depending on the real fluid pressure. Disturbances in the sensor data are reduced, allowing false alarms to be identified and minimized.

The present disclosure furthermore renders it possible to determine a second target variable in the form of a trend or a probability of an occlusion. Since some applications require a minimum pressure, the probability of an occlusion is also relevant in addition to the absolute pressure in the fluid conduit. According to the present disclosure, the probability of an occlusion is determined on the basis of the sensor data or input variables. A pressure increase on account of an occlusion follows a specific pattern in the sensor data. This pattern is identified by means of the AI unit in order to be able to identify an occlusion in timely fashion with the aid of a trend analysis, even in the case of low conveying rates. When conventional threshold value systems are used, several hours may elapse before an occlusion is identified and a corresponding alarm is triggered.

The distinction in relation to the absolute pressure or the first target variable may moreover be advantageous if high pressure in the application does not put the patient at risk but a swift alarm reaction is nevertheless desired. Specifically, to distinguish an occlusion from other influencing factors on part of the pump, the sensor data can be obtained, respectively at rest and during or just after the execution of a pump motor step. This puts the AI unit into a position of being able to distinguish a true occlusion of the fluid conduit from other disturbance variables, for example the repositioning of the patient.

The present disclosure furthermore renders it possible to determine a third target variable in the form of a location of an occlusion. This is based on the insight according to the present disclosure that the location of the occlusion, which for example describes a distance between the force measurement and the occlusion, causes a location-specific pattern in the sensor data.

Target variables are supplied to the alarm unit in order to allow the generation of alarms, pre-alarms or notifications for the user.

The AI unit uses statistical models and is trained via learning on the basis of data. This is referred to as “supervised learning”. This requires real sensor data or input data, which are linked to the target variables, that is to say for example “occlusion yes/no” and the real fluid pressure. That is to say, the target variables of the respective sensor data or input variables are known. Further sensor data or input data and the correlating target variables thereof can be produced by physical simulations. The AI unit learns using these data and is able to create a prediction model.

The data can be divided into training data and validation data in order to be able to subsequently validate the system and also verify whether the system has the required prediction accuracy. The AI unit is trained using the training data and subsequently tested using the validation data.

### BRIEF DESCRIPTION OF THE DRAWINGS

The present disclosure is described in detail below with reference to the drawings. In this case:

FIG. 1 very schematically shows an infusion system according to the present disclosure,

FIG. 2 very schematically shows an injection pump for use in an infusion system according to the present disclosure, and

FIG. 3 very schematically shows a peristaltic pump for use in an infusion system according to the present disclosure.

### DETAILED DESCRIPTION

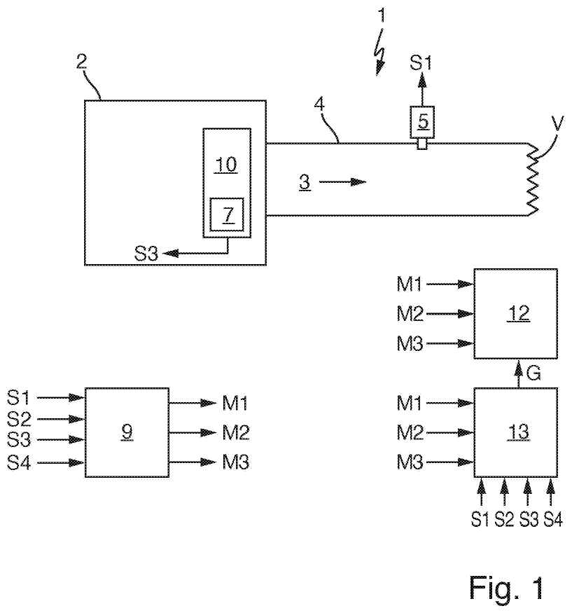

FIG. 1 very schematically shows an infusion system 1 according to the present disclosure comprising a conventional infusion pump 2, which is configured to convey a fluid 3 to be administered through a fluid conduit 4. In this respect, reference is also made to the relevant literature in the art.

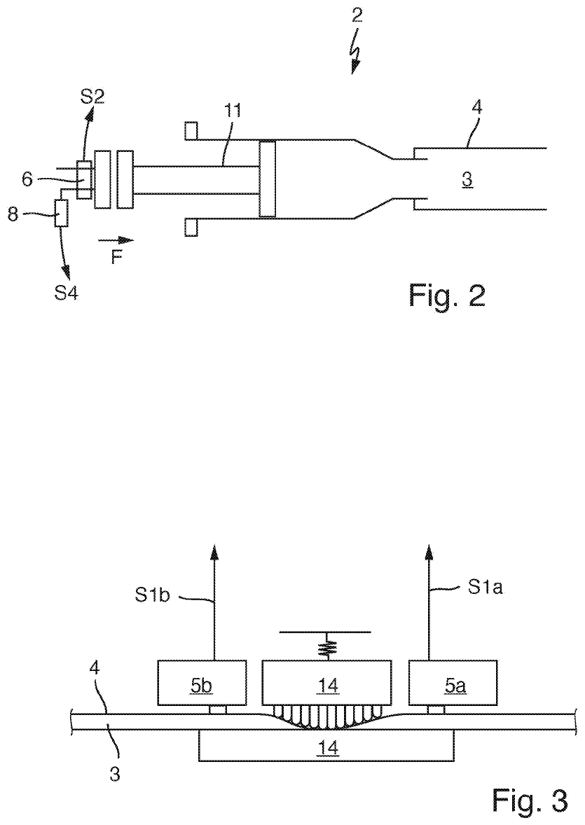

The infusion system 1 comprises further sensors 5, 6, 7, 8 (see also FIG. 2) for producing associated sensor data S1, S2, S3, S4. The sensor data 5, 6, 7, 8 can be analogue or digital sensor data.

The infusion system 1 further comprises an AI unit 9, which may be embodied as a microprocessor system in conjunction with suitable software, for example. The AI unit 9 is configured to calculate a first target variable M1, a second target variable M2 and a third target variable M3 depending on the number of sensor data S1, S2, S3, S4 using a statistical model as a basis, the statistical model having been trained in advance using training data.

The number of sensors comprise: a pressure sensor 5 configured to produce sensor data in the form of pressure data 51 in the fluid conduit 4 of the fluid 3 to be administered, a force sensor 6 (see FIG. 2) configured to produce sensor data in the form of force data S2 of a pump force F produced by means of the infusion pump 2, a motor current sensor 7 configured to produce sensor data in the form of motor current data S3 of an electric drive motor 10 of the infusion pump 2 and a position sensor 8 (see FIG. 2) configured to produce sensor data in the form of position data S4 of a syringe piston 11 of the infusion pump 2.

The number of target variables comprise: a first target variable M1 in the form of an absolute or real pressure in the fluid conduit 4, a second target variable M2 in the form of a probability of an occlusion V in the fluid conduit 4 and a third target variable M3 in the form of a location of the occlusion V in the fluid conduit 4.

The AI unit 9 may be configured to calculate the target variables M1, M2, M3 further depending on medicament data, patient data, data of further infusion pumps, therapy data, ambient data and/or vital parameter data using the statistical model as a basis.

Further, the infusion system 1 comprises an alarm unit 12 configured to verify on the basis of target variables M1, M2, M3 whether an alarm condition has been satisfied and produce an alarm should the alarm condition have been satisfied.

Further, the infusion system 1 comprises a correlation analysis unit 13 configured to calculate weight factors G on the basis of at least some of the sensor data S1, S2, S3, S4 and/or on the basis of at least some of the target variables M1, M2, M3, the alarm unit 12 being configured to verify also on the basis of the weight factors G whether the alarm condition has been satisfied. The alarm unit 12 is configured to stop infusion pump 2 when the alarm condition occurs.

FIG. 2 very schematically shows an infusion pump 2 in the form of a conventional injection pump for use in an infusion system 1 according to the present disclosure.

The force sensor 6 measures the resultant force F that is exerted on a piston plate of the syringe piston 11 of the infusion pump 2. This force F is composed of the force of the (fluid) pressure acting over the internal cross section of the syringe, that is to say the force of the first target variable M1, and disturbance variables superposed thereon. Disturbance variables include: position- and temperature-dependent static/dynamic friction effects, patient- and therapy-specific variables (diameter of the access, multiple infusions, type of access, movement as a result of repositioning, etc.), physical properties of the fluid to be conveyed (viscosity, temperature, etc.) and effects caused by ambient conditions (ambulance, helicopter, humidity, atmospheric pressure, etc.).

If there is an occlusion the pressure increases over the course of the conveyance. The increase in pressure is a function of the length of the fluid conduit 4 to the occlusion, that is to say the location of the occlusion or target variable M3, of the material employed and of the dimensions of the transmission system, the temperature and the conveyed volume once the occlusion occurs. An increasing force F on the drive head results in a greater motor current. As an alternative or in addition to the force sensor, said motor current can be detected and evaluated by means of the optional motor current sensor 7.

FIG. 3 shows a volumetric infusion pump or peristaltic pump 14, in which two pressure sensors 5a and 5b are arranged along the fluid conduit 4, each pressure sensor measuring the force exerted by the pressure in the fluid conduit 4 on the respective pressure sensor 5a and 5b, respectively, via the wall of said conduit. On account of peristalsis, there are different pressures on the upstream side (pump input/storage container) and downstream side (pump outlet/patient), and this is why two independent pressure sensors 5a and 5b, which generate the sensor data S1a and S1b, respectively, are used.

The force measured by means of the pressure sensors 5a and 5b is composed of the force of the fluid pressure acting of the internal cross section of the fluid conduit 4, that is to say the first target variable, and the disturbance variables superposed thereon. Disturbance variables include: temperature-dependent and use duration-dependent material properties, conditions on the upstream side (height and type of container, employed drip chamber, etc.), patient- and therapy-specific variables (diameter of the access, multiple infusions, type of access, movement as a result of repositioning, etc.), physical effects of the pumping principle (pattern during the normal conveyance, pattern as a result of mechanical pressure relief in the case of an occlusion, etc.), physical properties of the fluid to be conveyed (viscosity, temperature, etc.) and effects caused by ambient conditions (ambulance, helicopter, humidity, atmospheric pressure, etc.).

If there is an occlusion the pressure of the liquid increases over the course of the conveyance. The increase in pressure is a function of the length of the conduit to the location of the occlusion, i.e., the third target variable, of the material employed and of the dimensions of the transmission system, the temperature and the conveyed volume once the occlusion occurs.

According to the present disclosure, the overall system of a bay may be considered in addition to the sensor data S1 to S4 by virtue of all data of the involved infusion pumps, information about the medication and available patient data (e.g., from other medical equipment) being related to one another.

Examples of usable patient data include, e.g., vital parameters (blood pressure, pulse rate, respiratory frequency, body temperature, etc.) and further physiological parameters (oxygen saturation, blood sugar levels, blood count, size, age, weight, sex, hereditary factors, previous diseases, diagnoses, etc.). The properties of the conveyed fluids (medicament and its criticality, half-life/duration of effect, etc.) can be determined from the information about the medication.

The correlation analysis unit 13 describes interactions between the measured/evaluated variables and implements a correlation analysis of these variables. By way of example, this may inter alia allow an occlusion to be identified earlier, for example by virtue of identifying a correlation between too little of a medicament being conveyed and the other available patient data. An example would be a falling blood pressure and heart rate when adrenaline is administered.

Raising the alarm can be implemented more intelligently in the sense that the sensitivity level of the occlusion identification is increased if a correlation is present, and hence the alarm is raised earlier than provided for in the original settings. Moreover, the reverse case can avoid alarms in the case of uncritical predicaments, reducing the user's stress as a result of unnecessary alarms.

A change in certain vital parameters or specific threshold values being exceeded when certain medicaments/food are/is administered may indicate an occlusion. Alarm priorities can be reduced or alarm thresholds can be increased for uncritical medicaments/fluids. Interactions of various pumps at the same port can be taken into account in a dedicated fashion and during the occlusion identification.

The AI system for the bay may be realized as a central server application, which may also provide an interface to a cloud. The data obtained may subsequently be incorporated in the optimization of the occlusion identification within the scope of further developments, or customer-specific occlusion patterns may be set.

A specific bay is the transportation in an ambulance or rescue aircraft, in which input variables of the ambient conditions are present. In this case, navigation data may be used in order to compensate centrifugal forces, for example, which act on the fluid (curves/pitch), in the system.

What is claimed:

1. An infusion system comprising:
an infusion pump configured to convey a fluid to be administered through a fluid conduit;
one or more sensors for producing a number of sensor data; and
an AI unit configured to calculate a number of target variables depending on the number of sensor data using a statistical model as a basis, the statistical model having been trained using training data.

2. The infusion system according to claim 1, wherein the one or more sensors comprise one or more of:
pressure sensors configured to produce pressure data in the fluid conduit;
force sensors configured to produce force data of a pump force produced by the infusion pump;
motor current sensors configured to produce motor current data of an electric drive motor of the infusion pump; and
position sensors configured to produce position data of a syringe piston of the infusion pump.

3. The infusion system according to claim 1, wherein the number of target variables comprise one or more of:
an absolute pressure in the fluid conduit;
a probability of an occlusion in the fluid conduit; and
a location of the occlusion in the fluid conduit.

4. The infusion system according to claim 1, wherein the AI unit is configured to calculate the number of target variables based on one or more of medicament data, patient data, data from a second infusion pump, therapy data, ambient data and vital parameter data using the statistical model.

5. The infusion system according to claim 1, further comprising an alarm unit configured to verify whether an alarm condition is satisfied based on the number of target variables and to produce an alarm when the alarm condition is satisfied.

6. The infusion system according to claim 5, further comprising a correlation analysis unit configured to calculate weight factors based on at least one of:
at least one of the number of sensor data; and
at least one of the number of target variables,
wherein the alarm unit is further configured to verify whether the alarm condition is satisfied based on the weight factors.

7. The infusion system according to claim 5, wherein the alarm unit is configured to stop the infusion pump when the alarm condition is satisfied.
